(19) United States
(12) Patent Application Publication
(10) Pub. No.: US 2026/0083882 A1
(43) Pub. Date: Mar. 26, 2026

(54) HEAT SHRINK TUBE

(71) Applicant: Zeus Company LLC, Orangeburg, SC (US)

(72) Inventors: Zeth John Eberling, Lexington, SC (US); Tyler Austin Poole, Lexington, SC (US); Josh G. Fogle, Columbia, SC (US)

(21) Appl. No.: 19/086,581

(22) Filed: Mar. 21, 2025

Related U.S. Application Data
(60) Provisional application No. 63/696,924, filed on Sep. 20, 2024.
(60) Provisional application No. 63/772,234, filed on Mar. 14, 2025.

(51) Int. Cl. A61L 29/06; A61L 29/14

(52) U.S. Cl. CPC A61L 29/06; A61L 29/14

(57) ABSTRACT

The present disclosure provides tubings including at least one amorphous polymer resin. The tubings can exhibit heat shrink capability and, optionally, translucency or transparency through a wall of the tubing. After heating to recover the tubings, they can advantageously release cleanly from any underlying materials with which they are in intimate contact. The disclosure further provides methods of making and using such tubings and various applications for such tubings.

### CROSS-REFERENCE TO RELATED APPLICATIONS

The present application claims priority to and the benefit of U.S. Provisional Application Nos. 63,696,924, filed Sep. 20, 2024 and 63/772,234, filed on Mar. 14, 2025, the disclosures of which are both incorporated herein by reference in their entireties.

### FIELD OF THE INVENTION

The present application is directed to polymeric heat shrink tubing and methods for making such polymeric heat shrink tubing, which finds application in a variety of fields.

### BACKGROUND OF THE INVENTION

Heat shrink tubing generally comprises a plastic material that is extruded into a tubular form and expanded. The extruded and expanded tube is designed to shrink (i.e., decrease in diameter) when heated to a given temperature. As such, heat shrink tubing can serve various functions. It can provide a tight, protective jacketing to closely cover and insulate various elements (e.g., to protect them from abrasion and to provide thermal, chemical, moisture, and/or electrical insulation); it can serve to bundle certain elements together (i.e., within the same heat shrink tube); it can serve to seal/isolate certain elements from others; it can be used to join/fuse two elements, e.g., two tubes together; and it can serve to modify the properties of an underlying material (e.g., by closing around another material and shrinking that material as well). These capabilities render the tubing useful for various purposes and heat shrink tubing finds use across various fields, e.g., medical, chemical, electrical, optical, electronic, aerospace, automotive, and telecommunications fields.

In the medical context, heat shrink tubing is particularly beneficial in designing increasingly small and more complex devices to be inserted into the body (e.g., catheters, endoscopes, etc.). One representative medical use of heat shrink tubing is in the context of manufacturing a guide catheter, comprising a tubular structure having an inner layer of a polymer, a middle layer of a wire braid and an outer layer of another polymer. To assemble such catheters, an expanded heat shrink tube is typically applied to an assembled shaft around a mandrel and the assembly is exposed to high temperature sufficient to shrink the heat shrink tube. Under these conditions, the outer polymeric layers within the catheter shaft melt and flow, and the heat shrink tube contracts, providing compressive forces such that the inner and outer polymeric layers of the catheter shaft can bond together, encapsulating the wire braid within. The heat shrink tubing is then removed and discarded and the catheter assembly is removed from the mandrel. See, e.g., the disclosures of U.S. Pat. No. 7,306,585 to Ross and 5,755,704 to Lunn, which are incorporated herein by reference.

### SUMMARY OF THE INVENTION

Catheter shafts are typically manufactured by utilizing a fluorinated ethylene-propylene copolymer (FEP) heat shrink tubing. Such heat shrink tubing has been produced commercially for several decades using various processes, for example, vacuum expansion, gas pressure forming, sequential heating/stretching and the like. Known methods for expanding heat shrink tubing are provided, for example, in the disclosures of U.S. Pat. No. 2,987,767 to Edward et al.; U.S. Pat. No. 3,412,189 to Sullivan; U.S. Pat. No. 7,625,194 to Yoshida et al.; U.S. Pat. No. 9,296,165 to Henson; U.S. Pat. No. 9,327,444 to Henson; and U.S. Pat. No. 9,440,044 to Roof et al.; each of which is incorporated herein by reference. In most cases, the manufacturing process involves the melt extrusion of a polymer input tube defined by an inner diameter and outer diameter. The polymer input tube is then subjected to an expanding process to obtain the final heat shrink tube characterized by an expanded inner diameter and expanded outer diameter. The expanding process is typically carried using air or hydraulic pressure in the lumen of the input tube at an elevated temperature to expand the tube within the confines of a die or mold. Persons skilled in the art are able to manipulate the process parameters of temperature, dwell time and pressure to obtain the desired expanded tube.

It has long been believed that heat shrink tubing must have a melting temperature that greatly exceeds the melting temperature of the jacketing material for effective reflow in catheter manufacturing processes. See for example, J. Fry, “What you need to know about Heat Shrink Tubing”, Medical Design and Outsourcing, Jun. 22, 2017 [https://www.medicaldesignandoutsourcing.com/heat-shrink-tubing/]. The inventors have now discovered that there are two key parameters driving the effectiveness of the reflow process. The first parameter is the difference between the melting temperature of the jacketing material and the onset temperature of recovery of the heat shrink sleeve. The melting temperature of the jacketing material is measured by Differential Scanning calorimetry (DSC), while the onset temperature of recovery is measured by Dynamic Mechanical Analysis (DMA). The second parameter is the difference between the Vicat softening temperature of a heat shrink material and the onset temperature of recovery of the heat shrink sleeve. Softening temperature is measured according to ISO 306 (Vicat) Rate B/120. The use of these two parameters allows us to characterize amorphous materials, that have no well-defined melting temperature, as effective heat shrink tubes for catheter reflow. Currently, heat shrink tubing for catheter reflow processes are predominantly semi-crystalline in nature and include polymers such as fluorinated ethylene propylene (FEP), polytetrafluoroethylene (PTFE), polyethylene terephthalate (PET), polyether block amide (PEBA), polyamides and polyolefins. This discovery makes it possible to characterize amorphous materials, which have no well-defined melting temperature, as effective heat shrink tubes for catheter reflow.

It is also worth noting that, although heat shrink tubing is an essential feature of some final products, in many applications, it is removed prior to use of some final products such as sensor covers, strain reliefs, wire splicing aids, and particularly in medical applications. Often when used in medical applications, the heat shrink tubing is involved only in the manufacturing of the final product and is removed from the final product prior to use. Therefore, an additional step involved in the use of heat shrink tubing in certain applications, is removal of the heat shrink tubing from the underlying material. Removability of heat shrunk tubing following use thereof can be facilitated by a score line or indentations/perforations added prior or subsequent to use (i.e., heating) of the heat shrink tubing. After use, the heat shrink tubing can be torn along the scored line or indentations/perforations and discarded. Alternatively, a non-pre-scored heat shrink tube is scored down the length of the tubing following use (i.e., after being shrunk), and the tubing is then torn along the line and discarded. Accordingly, there is a need for tubing that can be applied to device components to encapsulate and compress them as needed, wherein the tubing can be readily and reliably removed with minimal potential to damage the underlying device components. Furthermore, the recovered heat shrink tubing must release from the underlying substrate without undue force.

Heat shrink tubing is widely used in industrial applications due to its ability to provide durable, flexible, and protective coverings in various environments. The tubing is applied by heating it, causing it to contract and form a tight seal around the object it encloses. This technology is particularly useful in applications such as sensor covers, strain reliefs, and wire splicing aids. In these instances, heat shrink tubing offers insulation, mechanical protection, and environmental sealing, shielding sensitive components from moisture, abrasion, and other external factors that could compromise functionality. The versatility of heat shrink tubing allows it to adapt to a wide range of shapes and sizes, making it a crucial component in ensuring the longevity and reliability of industrial systems.

The present disclosure relates to polymeric heat shrinkable tubes comprising one or more amorphous polymers that are not fluorinated. In certain embodiments, such tubes can comprise no more than one resin and in other embodiments, such tubes can comprise two or more resins (e.g., a main resin and one or more secondary resins). The resin or resins in these tubing can vary and can, in some embodiments, comprise a poly(etherimide-siloxane) copolymer (PEI-Sil) resin.

In some embodiments, at least one resin comprises one or more resins selected from the group consisting of poly(etherimide) (PEI), poly(imide) (PI), poly(carbonate) (PC), poly(siloxane), poly(sulfone) (PSU), poly(ethersulfonc) (PESU), poly(phenylene sulfone) (PPSU), and copolymers, blends, and derivatives thereof. For example, in one particular embodiment, the tubing may comprise a PEI-Sil copolymer resin as the main resin (e.g., in an amount of at least about 50% by weight) and one or more secondary resins selected from the list above.

In a further aspect of the disclosure is provided a tubing, comprising no more than one polymeric amorphous, melt processable resin, wherein the tubing exhibits heat shrink capability, and translucency or transparency through a wall of the tubing. In some embodiments, such a tubing can consist essentially of a single polymeric amorphous melt processable resin. Exemplary resins for such tubes include, but are not limited to, poly(etherimide) (PEI), poly(carbonate) (PC), poly(siloxane), poly(imide) (PI), poly(sulfone) (PSU), poly(ethersulfone) (PESU), poly(phenylene sulfone) (PPSU), and copolymers and derivatives thereof.

Typically, such heat shrink tubes are in the form of extruded and expanded tubes. Such heat shrink tubes can be subsequently employed during catheter shaft assembly processes as a manufacturing aid for catheter shafts. With propitious conditions of expansion, recovery ratio, and time/temperature profile for the recovery process, a satisfactory catheter shaft can be manufactured using a heat shrink tube comprising a non-crosslinked resin or resins (e.g., non-crosslinked PEI-Sil), which does allow for removal of the manufacturing aid from the catheter outer jacket post-recovery.

The disclosure provides, without limitation, the following embodiments:

Embodiment 1: A heat shrink tubing for recovery over a polymer substrate, wherein the heat shrink tubing comprises at least one amorphous polymer resin, wherein the heat shrink tubing exhibits a recovery temperature and a Vicat softening temperature and the polymer substrate exhibits a melting point such that: a) a first delta (Δ_{TO}) between the melting point of the polymer substrate and an onset point of the recovery temperature of the heat shrink tubing is a positive value; and b) a second delta (Δ_{VO}) between the Vicat softening temperature of the heat shrink tubing and the onset point of the recovery temperature of the heat shrink tubing is a positive value.

Embodiment 2: The heat shrink tubing of Embodiment 1, wherein the at least one amorphous polymer resin is a non-fluorinated amorphous polymer.

Embodiment 3: The heat shrink tubing of Embodiment 1 or 2, comprising no fluorinated polymer.

Embodiment 4: The heat shrink tubing of any of Embodiments 1-3, that exhibits translucency or transparency.

Embodiment 5: The heat shrink tubing of any of Embodiments 1-4, wherein the tubing comprises no more than one amorphous polymer resin.

Embodiment 6: The heat shrink tubing of Embodiment 5, wherein the no more than one amorphous polymer is selected from the group consisting of poly(etherimide) (PEI), poly(imide) (PI), poly(carbonate) (PC), poly(siloxane), poly(sulfone) (PSU), poly(ethersulfone) (PESU), poly(phenylene sulfone) (PPSU), polyetherimide siloxane (PEI-Sil) copolymer, and copolymers and derivatives of any one or more thereof.

Embodiment 7: The heat shrink tubing of any of Embodiments 1-4, wherein the tubing comprises two or more amorphous polymer resins.

Embodiment 8: The heat shrink tubing of Embodiment 7, wherein the two or more amorphous polymer resins comprise: a first amorphous polymer resin selected from the group consisting of poly(etherimide) (PEI), poly(imide) (PI), poly(carbonate) (PC), poly(siloxane), poly(sulfone) (PSU), poly(ethersulfone) (PESU), poly(phenylene sulfone) (PPSU), and copolymers, blends, and derivatives of any combination thereof; and a second amorphous polymer resin selected from the group consisting of poly(etherimide) (PEI), poly(imide) (PI), poly(carbonate) (PC), poly(siloxane), poly(sulfone) (PSU), poly(ethersulfone) (PESU), poly(phenylene sulfone) (PPSU), polyetherimide siloxane (PEI-Sil) copolymer, and other copolymers, blends, and derivatives of any combination thereof.

Embodiment 9: The heat shrink tubing of any of Embodiments 1-8, consisting essentially of the at least one amorphous polymer resin.

Embodiment 10: The heat shrink tubing of any of Embodiments 1-8, further comprising one or more additives.

Embodiment 11: The heat shrink tubing of any of Embodiments 1-10, wherein the at least one amorphous polymer resin is not cross-linked.

Embodiment 12: The heat shrink tubing of any of Embodiments 1-10, wherein the at least one amorphous polymer resin is at least partially cross-linked.

Embodiment 13: The heat shrink tubing of any of Embodiments 1-12, wherein the tubing exhibits longitudinal peelability.

Embodiment 14: The heat shrink tubing of Embodiment 13, comprising no physical score line or cut or embedded physical component to promote the longitudinal peelability.

Embodiment 15: The heat shrink tubing of Embodiment 13, comprising a nick no greater than about 1/50th of the length of the tubing to facilitate the longitudinal peelability.

Embodiment 16: The heat shrink tubing of any of Embodiments 13-15, wherein the longitudinal peelability does not require a physical score line or cut or embedded physical component to promote the longitudinal peelability or wherein the longitudinal peelability requires a physical score line, cut, or nick that is less than about 1/50th the length of the tubing.

Embodiment 17: The heat shrink tubing of any of Embodiments 1-16, wherein the tubing has an average wall thickness of less than 0.02 inches, e.g., 0.001 inches to 0.02 inches.

Embodiment 18: The heat shrink tubing of Embodiment 17, wherein the average wall thickness is 0.002 inches to 0.01 inches.

Embodiment 19: The heat shrink tubing of Embodiment 17, wherein the average wall thickness is 0.004 inches to 0.0099 inches.

Embodiment 20: A catheter assembly comprising the heat shrink tubing of any of Embodiments 1-19.

Embodiment 21: A construction, comprising a recovered heat shrink tubing comprising the heat shrink tubing of any of Embodiments 1-19 recovered over the polymer substrate, wherein the polymer substrate is in reflowed form.

Embodiment 22: The construction of Embodiment 21, wherein the polymer substrate exhibits a coefficient of variation (CoV) of its outer diameter (OD) of 0.005 or less.

Embodiment 23: The construction of Embodiment 21 or 22, wherein the recovered heat shrink tubing exhibits a longitudinal to radial percent shrinkage heat shrink ratio of 0.5 or less with respect to the heat shrink tubing.

Embodiment 24: The construction of any of Embodiments 21-23, wherein the recovered heat shrink tubing exhibits contact clarity through to the polymer substrate.

Embodiment 25: The construction of any of Embodiments 21-24, wherein the recovered heat shrink tubing has an average wall thickness of 0.001 to 0.028 inches.

Embodiment 26: The construction of Embodiment 25, wherein the average wall thickness is obtained when subjected to unrestricted recovery at 420° F. for five minutes.

Embodiment 27: A method of preparing a heat shrink tubing for recovery over a polymer substrate, comprising: selecting a heat shrink tubing material comprising at least one amorphous polymer resin that exhibits a recovery temperature and a Vicat softening temperature; and selecting a polymer substrate material that exhibits a melting point, wherein the recovery temperature, Vicat softening temperature, and melting point are such that: a) a first delta (Δ_{TO}) between the melting point of the polymer substrate and an onset point of the recovery temperature of the heat shrink tubing is a positive value; and b) a second delta (Δ_{VO}) between the Vicat softening temperature of the heat shrink tubing and the onset point of the recovery temperature of the heat shrink tubing is a positive value.

It will be apparent to those skilled in the art that other embodiments of the invention are possible and that the examples presented here are not intended to be exhaustive. These and other features, aspects, and advantages of the present disclosure will be apparent from a reading of the following detailed description together with the accompanying drawings, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended to be combinable, unless the context of the disclosure clearly dictates otherwise.

### BRIEF DESCRIPTION OF THE DRAWINGS

To provide an understanding of the embodiments of the invention, reference is made to the appended drawings, which are not necessarily drawn to scale, and in which reference numerals refer to components of exemplary embodiments of the invention. The drawings are exemplary only and should not be construed as limiting the invention.

FIG. 1 is a general schematic of a typical (but non-limiting) catheter relevant to certain embodiments of the disclosure, with relevant parameters, and an expanded schematic of one cross-sectional end face thereof;

FIG. 2 is a general schematic representation of a peelable tubing scored at one longitudinal end of the tubing according to certain embodiments of the disclosure; and

FIG. 3 is a general schematic of a non-limiting adhesion testing sample setup.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The present disclosure provides heat shrink tubes with unique properties and unique combinations of properties, as will be outlined further herein. Generally, a heat shrink tube is a shrinkable tubing prepared via expansion of a polymeric (“input”) tubing (e.g., an extruded tubing) to give the heat shrink tubing (also referred to herein as an “expanded” form). Upon heating, the heat shrink tubing “shrinks” to a size that is equivalent to (or close to) its original/input size, commonly referred to as its “recovered” size. The composition and overall size of a heat shrink tubing according to the present disclosure can vary widely and is not particularly limited. A heat shrink tubing can be defined, e.g., by measurable properties such as its inner diameter (“ID”) either after expansion (also referred to herein as “expanded inner diameter” (IDₑ)) or after recovery (also referred to herein as “recovered inner diameter” (IDᵣ)), its length (L), its change in length upon recovery (i.e., its percent change in length upon recovery, ΔL), its average wall thickness, its wall thickness concentricity (also referred to herein as percent concentricity or simply as concentricity), its expansion ratio (ER), its recovery ratio (RR), and its percent change in inner diameter upon recovery (ΔID). Such properties can be defined using the following equations:

$$Expansion ratio = ER = \frac{{ID}_{e}}{{ID}_{o}} \\ Recovery ratio = RR = \frac{{ID}_{e}}{{ID}_{r}} \\ \% Change in Length = Δ L = \frac{{L}_{r} - {L}_{e}}{{L}_{e}} ( 100 ) \\ \% Change of Inner Diameter = Δ ID = \frac{{ID}_{e} - {ID}_{r}}{{ID}_{e}} ( 100 ) \\ \% Concentricity = \frac{{wt}_{min}}{{wt}_{max}} ( 100 )$$

In these equations, Le and L are the length of the heat shrink tubing (in expanded form) and the length of the “recovered” (i.e., heat-shrunk) tubing, respectively. IDₒ refers to the original internal diameter (ID) of the input tube (i.e., the tube before it is expanded and then subsequently “shrunk”); IDₑ refers to the internal diameter (ID) of the expanded heat shrink tubing; and IDᵣ refers to the internal diameter (ID) of the recovered (heat shrunk) tube. Values needed for determination of percent concentricity are the minimum wall thickness and the maximum wall thickness of the tubular walls, defined as wtₘᵢₙ and wtₘₐₓ, respectively. RR, ΔL, and ΔID can be evaluated under any recovery conditions (i.e., time, temperature, and method of heat application), though the time and temperature at which an expanded tube is recovered must be specified as this can influence the observed extent of recovery (i.e., an expanded tube that is exposed to a lower temperature and/or for a shorter time may not recover to its full capability). Percent concentricity can be evaluated in the expanded or recovered state. Concentricity is a measure of wall thickness uniformity, and the concentricity value can influence performance in certain applications in both states. As used herein, the above parameters were calculated as follows.

The percent change in length (ΔL), also referred to herein as longitudinal change, is determined in the following manner. Prior to placing the heat shrink tubing into the oven for unrestricted recovery, the expanded tubing is cut to a length of 5 inches using a verified ruler. The 5 inch specimen length is carefully cut from the heat shrink tubing to ensure there are no burs or other deformities present, and that they are perpendicular to the longitudinal axis of the tubing. After the unrestricted recovery process at a specified temperature, the tubing length is re-measured using a verified ruler to the nearest 1/32nd of an inch to determine the amount of shrinkage or growth that has occurred during the process. For example, the expanded length is subtracted from the recovered length and divided by the expanded length, then this quantity is multiplied by 100 to give the overall percent change in length (ΔL) resulting from recovery. Typically, ΔL is measured to be in the range of about +/−15% (i.e., the length changes by less than about 15% upon recovery).

The recovery ratio (RR), percent change in inner diameter (ΔID), and percent concentricity is determined in the following manner. Specimens are cut from the expanded tubing and their expanded ID and wall thickness is measured using verified measurement tools. Multiple wall thickness measurements must be taken to accurately determine the percent concentricity (i.e., the wall thickness uniformity) of the tubular walls. The minimum wall thickness measurement taken on the expanded tube is divided by the maximum wall thickness measurement taken on the expanded tube and then multiplied by 100 to give the percent concentricity of the expanded tube. The specimens are then placed into an oven set at a specified temperature for approximately 10 minutes. After exposing each heat shrink tubing specimen to a specified recovery temperature for 10 minutes, it is removed from the oven and allowed to cool to ambient temperature. This subjects the expanded heat shrink tubing to an unrestricted recovery process. After cooling to ambient temperature, the recovered ID and wall thickness is measured using verified measurement tools. The expanded tubing ID is divided by the recovered tubing ID to calculate the recovery ratio (RR) of the heat shrink tube under the specified recovery conditions (i.e., recovery temperature and time). Subsequently, the percent inner diameter change of the heat shrink tubing is calculated by subtracting the recovered tubing ID from the expanded tubing ID and dividing by the expanded tubing ID, then multiplying this quantity by 100 to give the overall percent change in inner diameter (ΔID). The minimum wall thickness measurement taken on the recovered tube is divided by the maximum wall thickness measurement taken on the recovered tube and then multiplied by 100 to give the percent concentricity of the recovered tube.

The percentage radial shrinkage (ΔR) is determined using the below formula. % Radial Shrinkage (ΔR)=(1−1/RR)*100. The heat shrink ratio of longitudinal to radial shrinkage (ΔL/ΔR) is calculated using the formula found below.

$$Δ R = ( 1 - \frac{1}{RR} ) \times 100 Ratio of \% longitudinal shrinkage to \% radial shrinkage = \frac{Δ L}{Δ R}$$

Certain properties of the heat shrink tubings disclosed herein can be evaluated by utilizing an oven reflow test. In this test a laboratory forced air oven is utilized to recover the example heat shrink tubes over the top of different polymer substrates (e.g., jacket polymer substrates) which are supported by a mandrel.

Test specimens are prepared by first selecting a mandrel which has an outer diameter 0.005 to 0.035 inches greater than the recovered ID of the heat shrink to be tested. Next a thin walled, OD etched PTFE tube with an ID 0.001 to 0.002 inches greater than the OD of the mandrel and a wall thickness between 0.001 and 0.003 inches is slid over the mandrel. The PTFE tube is stretched down to form a snug fit with the mandrel and the ends are secured to prevent movement. A thermoplastic polymer tube with an ID 0.006 to 0.020 inches greater than the OD of the PTFE covered mandrel, a wall thickness between 0.003 and 0.010 inches, and a length that is at least 2 inches less than the mandrel length is dried per vendor recommended drying conditions. Once dry, the thermoplastic tube is slid over the top of the PTFE covered mandrel and centered down the length. The example heat shrink tube is cut to a length to allow for at least a 0.5 inch overhang of the ends of the thermoplastic tube. The example heat shrink tube is slid over the top of the thermoplastic tube and centered down the length.

The prepared specimen is then positioned vertically in a forced air laboratory oven set to 420° F. for a period of 5 minutes. After which the specimen is removed from the oven and allowed to cool. Once cooled to room temperature the example heat shrink is removed from the exterior of the test specimen, taking care to not damage the substrate. The mandrel is then removed from the ID of test specimen, taking care to not damage or deform the test specimen.

The OD of the test specimen is measured in at least 3 separate locations down the length, avoiding the first and last inch of the test specimen. A 3-axis laser OD micrometer is utilized to perform these OD measurements, and each measurement is the average of the 3 axes. From these measurements, the Average OD of the test specimen is calculated along with the OD standard deviation. The OD standard deviation is divided by the Average OD to calculate the Coefficient of Variation (CoV).

In some embodiments, a first delta (Δ_{TO}) is the difference between the melting point temperature (T_{M}) of the jacket polymer substrate and the recovery onset temperature of the heat shrink material. The first delta (Δ_{TO}) is calculated by subtracting the heat shrink recovery onset temperature (° C.) from the melting point temperature (T_{M}) (° C.) of the jacket polymer substrate

In some embodiments a second delta (Δ_{VO}) is the difference between the Vicat softening temperature (° C.) and the recovery onset temperature of the heat shrink material (° C.). The second delta (Δ_{VO}) is calculated by subtracting the heat shrink recovery onset temperature (° C.) from the Vicat softening temperature (° C.) of the heat shrink resin. In some embodiments when both of these delta values (Aro and Avo) are positive, heat shrink tubes can be manufactured with excellent reflow properties over a variety of polymer substrate jacket materials such as PEBA, polyamides, polyurethanes, thermoplastic elastomers, polyolefins, and polyesters. This list is meant to be non-limiting.

By carefully selecting amorphous heat shrink tubing materials and by tailoring the input tube and expansion process conditions such as expansion air temperature, pressure, and dwell time, stress imparted on the materials can be optimized to achieve a positive Δ_{TO} between the heat shrink recovery onset temperature and the melting points of any jacket polymer substrate (e.g. PEBA, polyamides, polyurethanes, thermoplastic elastomers, polyolefins, and polyesters, and other conventionally used jacket polymer substrate materials ranging from soft to hard durometers and high to low melting points) and achieve a positive Δ_{VO} between the Vicat softening temperature of the heat shrink material and the heat shrink recovery onset temperature.

To evaluate the quality of the reflow, the test specimen is visually inspected down its length, avoiding the first and last inch of the test specimen, for defects such as bubbles, lines, dimples, voids, and lumps. The visual inspection takes place with the unaided eye at a distance of 12-18 inches. The test specimen is assigned a numeric grade of 0-3 depending on the number and severity of the defects. A grade of 0 is free from any such defects in the inspection region. A grade of 1 has a very limited number of defects that are observed on less than ⅓ of the inspection region's length. A grade of 2 has defects that are observed on more than ⅓ and less than ⅔ of the inspection region's length. A grade of 3 has defects that are observed on more than ⅔ of the inspection region's length. In the event that a particular defect or set of defects is considerably egregious, but not covering more than ⅔ of the inspection region's length, a grade of 3 is still utilized.

A good quality tube will have an OD CoV less than about 0.005 or 0.05%, and the absolute value of the ratio of percent longitudinal shrinkage to percent radial shrinkage will be less than about 0.5.

In some embodiments, the disclosed heat shrink tubes are amorphous, non-fluorinated heat shrink tubes. Such tubes can comprise, consist essentially of, or consist of one or more polymers such as polyethers, polyimides, polycarbonates, polysulfones, polysiloxanes, polyetherimide siloxanes or a copolymer, blend, or derivative of any two or more of the foregoing. One non-limiting polymer for use in certain non-crosslinked heat shrink tubes (alone or in combination with one or more other polymers) according to the present disclosure is a poly(etherimide-siloxane) (PEI-Sil) (i.e., a copolymer of polyetherimide and polysiloxane).

In some embodiments, the disclosed amorphous, non-fluorinated heat shrink tubes comprise, consist essentially of, or consist of one or more amorphous polymers and as such can be described based upon their Vicat Softening Temperature (ISO 306). In certain specific embodiments, the Vicat softening temperature is greater than about 70° C., is greater than about 110° C., is greater than about 160° C., is greater than about 170° C., or is greater than about 180° C., and typically less than about 300° C.

In certain specific embodiments, amorphous, non-fluorinated heat shrink tubes comprising PEI-Sil are provided. The ratio of the polyetherimide and polysiloxane of the PEI-Sil in the heat shrink tube can vary greatly without departing from the present disclosure. As such, in some embodiments, a given PEI-Sil can comprise a higher number of etherimide monomers than siloxane monomers; in some embodiments, a given PEI-SIL can comprise roughly equivalent numbers of etherimide and siloxane monomers; in some embodiments, a given PEI-Sil can comprise a higher number of siloxane monomers than etherimide monomers. Different composition ratios of the polyetherimide to polysiloxane influence the physical properties of the supplied resin; and ultimately the final physical properties of the non-crosslinked PEI-Sil heat shrink tubes of the present disclosure.

In various embodiments, the heat shrink tubings disclosed herein are prepared from one or more poly(etherimide-siloxane) (PEI-Sil) resins. “Resin” as used herein refers to a material consisting essentially of a given type of polymer (e.g., a copolymer) or two or more polymers/copolymers. Resins are typically provided in solid form (e.g., as solid pellets), although they are not limited thereto (with other forms including, but not limited to, powders, pastes, granules, dispersions, solutions, gels, and the like). In some embodiments, the heat shrink tubing disclosed herein may be prepared from a resin comprising, consisting of, or consisting essentially of a PEI-Sil resin in one or more of the forms noted herein. In some cases, a “resin” as used herein may contain one or more additional components as additives and/or one or more additional components can be added thereto (e.g., such as a lubricant, colorant, filler, and the like). In other embodiments, one or more additional components (in granular, powder, or pellet form or in the form of a gel or liquid) can be included with the PEI-Sil resin and extruded therewith. As such, the heat shrink tube ultimately produced can comprise, in some embodiments, one or more such additional component(s).

In certain embodiments, the heat shrink tubes of the present disclosure are prepared using a PEI-Sil resin, and thus in some embodiments, can consist of PEI-Sil, can consist essentially of PEI-Sil, or can comprise PEI-Sil. Typically, PEI-Sil resins can be provided in a variety of different forms, for example, in the forms of solid pellets, powders, granules, dispersions, solutions, gels, and the like, and any such form can be used according to the present disclosure.

In certain embodiments, heat shrink tubes are prepared using extrusion grade PEI-Sil resin pellets. The type of PEI-Sil resin that is utilized in certain embodiments can vary and may include PEI-Sil extrusion grade pellets of different compositions (i.e., different flexural modulus), either as a single PEI-Sil copolymer resin grade, as a blend of two or more PEI-Sil copolymer resin grades, as a coextrusion of two or more PEI-Sil copolymer resin grades, or as a blend that includes a PEI-Sil copolymer resin grade. The PEI-Sil resins utilized in certain embodiments may also be blended or compounded with other polymeric components (e.g., including, but not limited to, polycarbonate (PC)) to tailor the final properties of the resulting heat shrink tube for a particular application. Exemplary extrusion grade PC resins suitable for use according to the present disclosure are commercially available as LEXAN (T_{M}) Copolymer. Exemplary extrusion grade resins include, but are not limited to, LEXAN (T_{M}) XHT3143T and LEXAN™ HPH4504H. Exemplary extrusion grade PEI-Sil resins suitable for use according to the present disclosure, include but are not limited to, commercially available SILTEM® STM1500, SILTEM® STM1600, and SILTEM® STM 1700 manufactured by SABIC. However, it is to be understood that the heat shrink tubes provided herein are not limited to comprising or being prepared from PEI-Sil resins and may be prepared using one or more of the polymeric resins described herein in addition to PEI-Sil, or instead of PEI-Sil. Coextrusions of two or more resin families, for example PC and PEI-Sil are also suitable for use according to the present disclosure.

In some embodiments, one or more additives can be incorporated within the bulk of the heat shrink tubing walls. In some such embodiments, the one or more additives can be distributed (e.g., substantially uniformly) throughout the wall thickness and length of the tubing. In some embodiments, the one or more additives may include a lubricant, e.g., such as a thermally stable extrusion process lubricant. In some embodiments, the one or more additives may include a pigment to provide a desired color of the heat shrink tube. In some embodiments, other additives such as inert fillers, stabilizers (i.e., radiation stabilizers, antioxidants, etc.), conductive fillers, anti-tack agents and/or antimicrobials may be included to produce desired functionality of the heat shrink tube for specific applications. The amount of additive that can be contained in the heat shrink tube is not particularly limited. In some embodiments, for example, the one or more additives (e.g., lubricant, pigment, filler, etc.) may be included in an amount in the range of 0.1% to 80%, about 1% to about 30%, or about 5% to about 20% by weight based on the total weight of the heat shrink tube. In other embodiments, the heat shrink tubes may not include any additives therein.

The sizes of heat shrink tubes within the scope of this disclosure (e.g., length, diameter (i.e., expanded inner diameter, ID), and average wall thickness) are not particularly limited. For example, the length of tubes described herein can vary from individually sized units (e.g., in some embodiments, on the order of 0.1 inches to 120 inches for catheter or medical device component manufacturing) to lengths that can readily be transported and further cut into individually sized units to large-scale production lengths (e.g., on the order of hundreds of feet and the like). The diameters of tubes described herein can vary, in particular, depending upon the application for which the tubing is intended. Certain expanded IDs of the tubes described herein, particularly for catheter and medical device uses, can range from about 0.005 inches to about 1.5 inches (e.g., about 0.01 inches to about 0.7 inches or about 0.015 inches to about 0.5 inches), although tubes having expanded IDs outside this range are also encompassed by the present disclosure, particularly in the context of applications in different fields.

In general, the methods by which heat shrink tubes are prepared can vary. Generally, the desired resin or resins, such as the PEI-Sil resins as described herein, are converted into a tubular form via extrusion and then mechanically expanded. The means by which these steps are conducted can vary, as will be described herein.

A resin (e.g., such as a PEI-Sil resin) may be formed into a tube by subjecting the resin to extrusion. Extrusion generally comprises placing the desired resin or resins into an extruder (e.g., a single screw melt extruder). Within the extruder, the resin or resins are heated, compressed, and conveyed out of the extruder and through an extrusion head (also referred to herein as a “head”) containing an annular die set, creating a tube. The annular die set (also referred to herein as “tooling”) consists of a circular extrusion die and a mandrel which forms the polymer melt into a tubular form as it exits the extrusion head. Tubes of various diameters, wall thicknesses, and lengths can be produced using the forming methods described herein. The final dimensions of the extruded tubular form can be adjusted and optimized through proper tooling selection along with other parameters in the extrusion step such as temperature, pressure, and screw rotation speed. The extruder is generally comprised of a hopper, barrel, screw, breaker plate, and extrusion head. The screw of the extruder is generally comprised of several sections (e.g., the feed, compression, and metering zones) that can be optimized to provide an effective and consistent extrusion process. Generally, there are multiple temperature-controlled zones throughout the extruder, each of which can be adjusted and optimized to produce tubular forms of desired dimension and quality. In some embodiments, tubing having a relatively uniform wall thickness (i.e., high percent concentricity) is provided.

Appropriate sizing of the tooling to be used during the extrusion process is generally determined by the specified finished tubular dimensions, individual extruder specifications, the desired draw down ratio (DDR), and the draw ratio balance (DRB). The DDR and DRB are unitless quantities used by those skilled in the art to describe the relationship between the dimensions of the polymer melt as it exits the tooling to the dimensions of the final tubular form (i.e., the final extruded tube dimensions). Draw down ratio (DDR) is defined as the ratio of the cross-sectional area of the polymer melt as it exits the tooling to the cross-sectional area of the final tubular form. The molten tubular form must be “drawn down” (i.e., reduced in diameter and cross-sectional area via stretching) after exiting the tooling and before quenching (i.e., rapidly cooling in air or a chilled fluid bath) to obtain the desired final tubular dimensions. Generally, utilizing tooling that provides a high DDR with respect to the dimensions of the final tubular form enables faster line speeds (i.e., a faster production rate). It is also important to note that a tube produced with a higher DDR has a greater degree of longitudinal orientation of the polymer chains than a tube produced using a lower DDR. It is well known to those skilled in the art that imparting a particular degree of orientation on a polymeric material can influence the mechanical and physical properties of the final product. These properties (e.g., such as mechanical properties) can be optimized for a particular application (i.e., input for a secondary expansion process) by varying the degree of orientation. Draw ratio balance (DRB) is defined as the diameter ratio of the extrusion die and mandrel divided by the diameter ratio of the final tubular form. Generally, DRB characterizes the relationship between the annular shape of the polymer melt as it exits the die and the annular shape of the final tubular form. It is well known by those skilled in the art that careful tooling selection is required to achieve a stable tubular extrusion process. The dimensional relationship of the tooling used to produce a tube of a given size influences the line speed of the extrusion process (i.e., the production rate) based on the DDR. However, this dimensional relationship must be balanced because it also directly influences the overall stability of the extrusion process through the DRB. Tooling selection is an important aspect of the extrusion process to produce a tubular form of specified dimension that also has desirable mechanical characteristics.

The extruded tubular form is then typically radially expanded (e.g., by mechanical means) to provide an expanded tube, i.e., a heat shrink tube (i.e., a tube which decreases in diameter when heated). The expansion of the input tubing (i.e., the initial extruded tubular form) can be conducted in-line with extrusion or off-line (i.e., conducted independently of and/or secondary to the extrusion process). All means for radial expansion of tubing are intended to be encompassed by the present invention. Generally, during the expansion process, the tubing is expanded radially by pressurizing the inside of the tubing, introducing stress into the tube wall. This pressurizing can be conducted by any means capable of providing a differential pressure between the inside and outside of the tubing. Such differential pressure can be created by imposing a pressure above atmospheric pressure on the inside of the tube, imposing a pressure below atmospheric pressure on the outside of the tube, or a combination of the two. The stress induced into the wall of the tube causes it to expand radially, i.e., increase in diameter. The rate of expansion can be controlled so the tube will hold the expanded state and does not recover until subjected to a further heat cycle. The extent to which a tube is expanded depends on the application for which the final heat shrink tubing is intended. The rate and extent to which a tube is expanded depends on the temperature at which the expansion process is conducted. It has been found that the expansion chamber temperature must be carefully controlled to optimize the rate and extent of expansion of the tube. In some embodiments, the tubing is expanded to an inner diameter from about 1.05 times its original (unexpanded) inner diameter to about 10 times its original (unexpanded) inner diameter.

In certain embodiments, PEI-Sil heat shrink tubes prepared according to the present disclosure may be radially expanded using the processes described, for example, in U.S. Pat. No. 9,296,165 to Henson, which is incorporated by reference herein in its entirety. For example, the Henson patent describes a process to produce thermoplastic polymeric heat shrink tubing using a first fluid in the interior of a tube to expand it and a second fluid exterior to the tube to constrain the expansion within an expansion chamber. In other embodiments, for example, the tubing may be expanded by adjusting the flow rate of the air external to the tube, the chamber temperature, the air pressure within the tube, and/or the rate at which the tube moves through the expansion chamber. In certain embodiments, the heat shrink tubes of the present disclosure are expanded at elevated temperature through a die using any number of methods known to the art, and subsequently cooled at the die exit. Cooling can be accomplished using fluids such as water, oil, or air. The processing parameters that can be adjusted include, but are not limited to, die type, die diameter and length, die temperature, fluid pressure inside the tube, fluid pressure outside the tube, cooling method, cooling medium type and temperature, expansion rate, tube materials, tube ID, tube OD, and tube wall thickness.

Advantageously, as referenced herein, the disclosed heat shrink tubes are described as “amorphous” or as “exhibiting little to no crystallinity” and/or “non-fluorinated,” and in some embodiments, as being “amorphous, non-fluorinated heat shrink tubes.” In some embodiments by “amorphous” is meant there is no discernable melt endotherm on a differential scanning calorimetry (DSC) thermogram.

In some embodiments, “little to no crystallinity” means that less than 5%, less than 4%, less than 3%, less than 2%, less than 1%, less than 0.5%, less than 0.1%, or less than 0.01% of the material is in crystalline form, detectable, e.g., via differential scanning calorimetry (DSC).

In some embodiments, “little to no fluorinated material” means that no fluorinated material is intentionally added to/incorporated in the heat shrink tube (although this does not preclude the inclusion of small amounts of e.g., fluorine-containing contaminants that may or may not be present within the heat shrink tube). For example, in some embodiments, “little to no fluorinated material” means less than about 500 ppm of fluorine within the heat shrink tube, e.g., less than about 250 ppm, less than about 100 ppm, less than about 50 ppm, less than about 10 ppm, less than about 5 ppm, or less than about 1 ppm of fluorine. In some embodiments, “little to no fluorinated material” means that no detectable quantity of fluorine is present (e.g., via methods such as combustion ion chromatography, pyrolysis gas chromatography-mass spectrometry, or fluorine nuclear magnetic resonance spectroscopy).

Advantageously, as referenced herein, such heat shrink tubes can be provided wherein a majority (e.g., the entirety) of the polymer or polymers (e.g., PEI-Sil) within the tube is not cross-linked. In some embodiments, the tubes provided herein comprise no cross-linked polymer, comprise less than 2% by weight of a crosslinked polymer, less than 1% by weight of a cross-linked polymer, or less than 0.5% by weight of a cross-linked polymer.

In some embodiments, heat shrink tubes are provided wherein a majority (e.g., substantially the entirety or the entirety) of the polymer or polymers within the tube is cross-linked. In some embodiments, the tubes provided here comprise 10% by weight or more of cross-linked polymer, 20% by weight or more of cross-linked polymer, 50% by weight or more of cross-linked polymer, 80% by weight or more of cross-linked polymer, or 90% by weight or more of cross-linked polymer.

The disclosed heat shrink tubes can exhibit high recovery ratios; in some embodiments, the disclosed heat shrink tubes can have recovery ratios (RRs) of greater than about 1.05:1, greater than about 1.10:1, greater than about 1.5:1, greater than about 1.8:1, greater than about 2:1, greater than about 3:1, or greater than about 4:1, e.g., about 1.05:1 to about 4:1. In some embodiments, the heat shrink tubes can be described based on the reducibility in ID (upon recovery). Examples of such values include, but are not limited to, tubes reducible in ID by at least about 4%, at least about 9%, at least about 33%, at least about 44%, at least about 50%, at least about 66%, or at least about 75%, such as about 4% to about 75%.

With regard to the translucency or transparency of certain tubes disclosed herein, in certain embodiments, the tubes exhibit translucency through the wall of the tubes. Translucency is understood to mean that light passes through the tubing wall but diffuses to some extent. In some embodiments, the tubes exhibit transparency through the wall of the tubing. Transparency is understood to mean that light passes through the tubing wall and does not diffuse to any significant extent.

The translucency and/or transparency of the tubing walls disclosed herein can be described by the total light % transmittance through the wall, the diffuse light % transmittance through the wall, and the haze %. Total light % transmittance compares the intensity of the light entering a sample with the intensity of the light leaving the sample. If a sample absorbs no light, the intensity of light entering the sample is equal to the intensity of the light leaving the sample, i.e., total light % transmittance is 100%. By contrast, if a sample absorbs the light completely, the intensity of light leaving the sample is 0, i.e., transmittance is 0%. Diffuse light % transmittance relates to the scattering of light entering the sample by comparing the intensity of light entering a sample at a given angle to the intensity of light leaving that sample at that same angle. Haze % is the ratio of diffuse % transmittance to total % transmittance. If a sample allows all light to pass through with the angle unchanged, the diffuse light % transmittance is 0%, the haze is 0%, and the sample is considered transparent. If, however, a sample diffuses any portion of the light entering the sample, the diffuse light transmittance is greater than 0%, the haze is above 0%, and the sample is not transparent (but may still be translucent). Relevant measurements can be made, for example, using a hazemeter or spectrophotometer, using methods known in the art (e.g., ASTM D1003-13, “Standard Test Method for Haze and Luminous Transmittance” (2015) and ASTM D1746-09, “Standard Test Method for Transparency of Plastic Sheeting” (2015), which are incorporated herein by reference).

The total light transmittance of certain tubes provided herein is advantageously at least about 70%, at least about 75%, or at least about 85%. The diffuse light transmittance is advantageously less than about 25%, less than about 20%, or less than about 15%. Based on these values, tubes described herein can, in some embodiments, be described as exhibiting low haze through the tubing wall, e.g., being substantially free of haze. In some embodiments, the tubes exhibit haze of less than about 50%, less than about 40%, less than about 30%, or less than about 20%. In some embodiments, the tubes can be described as being substantially (e.g., completely) free of haze, e.g., having a haze of less than about 15%, including less than about 12% and at least about 10%.

Consequently, the tubing may allow for users to readily see the underlying material when the heat shrink tubing is applied in use. For example, in some embodiments, the tubing exhibits light transmission through the tubing wall of at least about 70%, at least about 80%, at least about 90%, at least about 98%, or at least about 99% of available light. However, the tubing is not limited thereto and in certain embodiments, can be colored (e.g., via the incorporation of dyes or colorants) and/or somewhat less translucent and/or opaque.

Advantageously, the light transmittance values through the walls of the tubes disclosed herein are significant at all wavelengths within the visible range (i.e., about 400 nm to about 750 nm). For example, in certain embodiments, the total light % transmittance through the wall of a given tubing is at least about 25% across the full visible spectrum. In certain embodiments, the total light % transmittance through the wall of a given tubing is at least about 30%, at least about 35%, at least about 40%, at least about 45%, or at least about 50% across the full visible spectrum. All optical properties referenced herein relate to testing a single, flat tubing wall of substantially customary thickness for heat shrink tubes (e.g., having wall thicknesses as disclosed above).

In some embodiments, the shrink tubes provided herein can exhibit good contact clarity once reflowed over the jacket polymer substrate material. Contact clarity in this context may include the visibility through the recovered heat shrink tube of important features of the underlying jacket polymer substrate. Important features can include weld joints, marker bands, surface flaws, color, images or writing, for example. Obscured visibility through the recovered heat shrink tube of the underlying jacket results in a contact clarity “fail” evaluation, while a “pass” evaluation of contact clarity is defined by the underlying jacket being readily visible to the unaided eye at a distance of 12-18 inches through the recovered heat shrink tube.

With regard to the ability to tear longitudinally (also referred to as “peelability” or simply “peel”), in certain embodiments, tubing provided according to the present disclosure is peelable lengthwise/longitudinally without use of any score lines, perforations, indentations, embedded component(s) like thread/string/wire or the like. In certain such embodiments, a small nick, cut, or tear may be made at one end of the tubing to facilitate peeling of the tubing longitudinally (e.g., by hand). In other embodiments, no such nick, cut, or tear is required, and the tubing can be readily peeled (e.g., by hand) by pulling apart two sides of the tubing, beginning at one end of tubing. In certain embodiments, the tubing described herein may exhibit one or more of complete, straight, and even peeling along a given length of the tubing.

For example, in some embodiments, the tubing provided herein can exhibit one or more of complete, straight, and even peeling along at least about 1 meter of tubing, at least about 10 meters of tubing, or at least about 100 meters of tubing. In some embodiments, the tubing is cut into individual lengths, such as into individual tubes (e.g., with lengths tailored to particular applications). Of course, it is understood that sizes (both diameters and lengths) can be tailored for different applications and may be substantially larger or smaller than the examples noted herein.

In one particular embodiment, tubes provided according to the present disclosure can be scored, cut, or nicked across the cross-section of the tubing diameter at one end, (providing a small score line or nick of about ½ inch or less in length) grasped (e.g., between the fingers or automated grips) and peeled without breaking. In some embodiments, the “peelability” or “tearability” can be achieved without any significant scoring or nicking. For example, in some embodiments, the score or nick across the cross-section of the tubing diameter has a length that is less than about 1/10th the length of the tubing to be peeled, less than about 1/25th the length of the tubing to be peeled, less than about 1/50th the length of the tubing to be peeled, or less than about 1/75th the length of the tubing to be peeled. In some embodiments, such values can allow for complete peeling of the entire length of tubing and the peeled halves of tubing can be substantially equal in size (i.e., the tubing exhibits complete, straight, and/or even peeling along the entire length of the tubing.

In some embodiments, the tube is substantially homogeneous throughout the wall and around the circumference of the tube, in terms of composition and thickness. For example, in some embodiments, the tube does not comprise any other component therein, e.g., embedded therein, such as a braid or other component to facilitate peeling of the tube after use.

The peel strength of the tubing materials described herein can vary. It is noted that preferred peel strengths vary with tubing diameter, with generally higher peel strengths preferred for larger diameters.

With regard to the longitudinal peelability, in certain embodiments, tubing provided according to the present disclosure is peelable lengthwise/longitudinally without use of any score lines, perforations, indentations, or the like. In certain such embodiments, a small nick, cut, or tear may be made at one end of the tubing to facilitate peeling of the tubing longitudinally (e.g., by hand). In other embodiments, no such nick, cut, or tear is required, and the tubing can be readily peeled (e.g., by hand) by pulling apart two sides of the tubing, beginning at one end of tubing. In certain embodiments, the tubing described herein may exhibit one or more of complete, straight, and even peeling along a given length of the tubing.

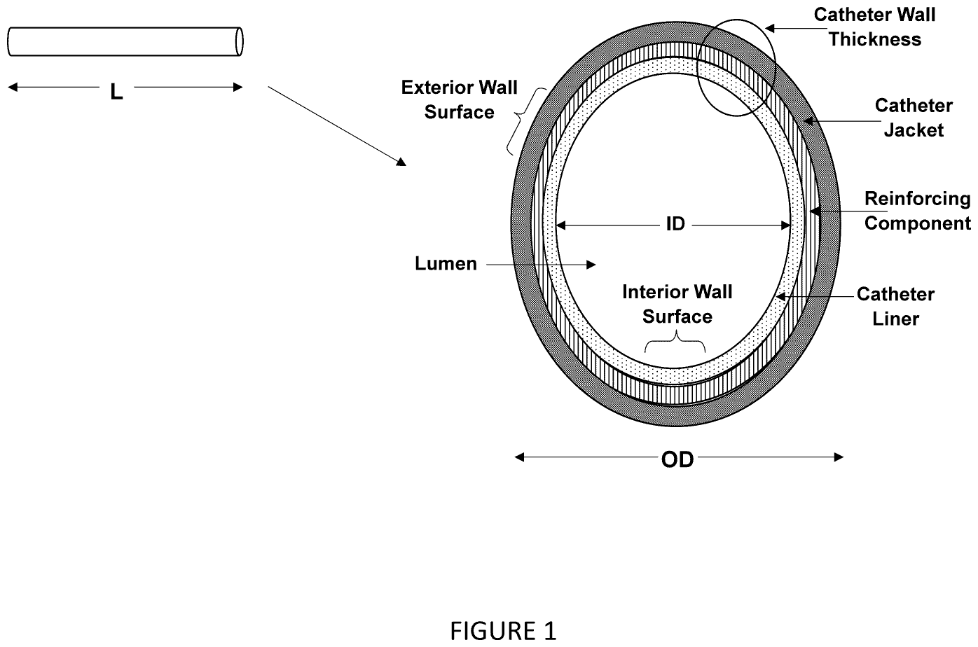

For example, in some embodiments, the tubing provided herein can exhibit one or more of complete, straight, and even peeling along at least about 1 meter of tubing, at least about 10 meters of tubing, or at least about 100 meters of tubing. In some embodiments, the tubing is cut into individual lengths, such as into individual tubes (e.g., with lengths tailored to particular) applications). Of course, it is understood that sizes (both diameters and lengths) can be tailored for different applications and may be substantially larger or smaller than the examples noted herein. In certain embodiments, such tubes can be peeled completely and substantially evenly along their full lengths, as shown in FIG. 1, where the tubing is peeled, e.g., from end A to end B of the tubing to give two substantially equal longitudinal “halves” of tubing.

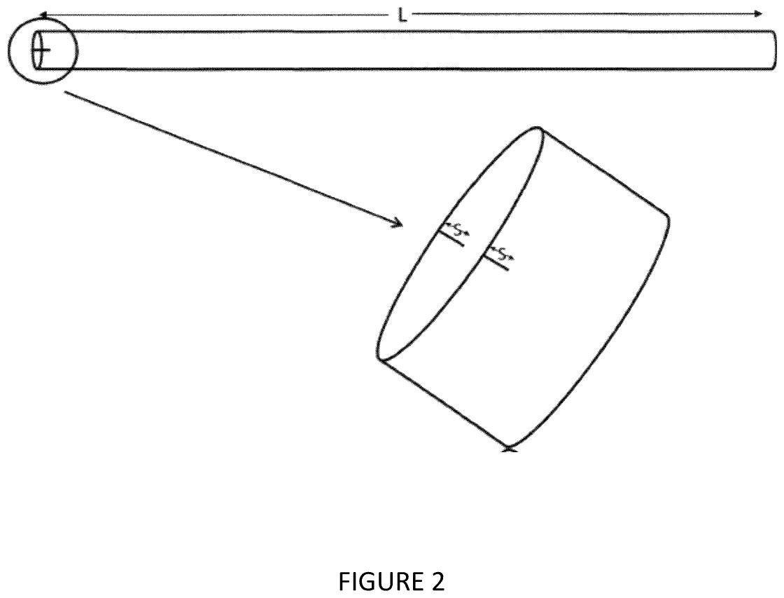

In one particular embodiment, tubings provided according to the present disclosure can be scored, cut, or nicked across the cross-section of the tubing diameter at one end, as shown in FIG. 2 (providing a small score line or nick of length “S,” e.g., about ½ inch or less in length). grasped (e.g., between the fingers or automated grips) and pulled/peeled without breaking or deviating from a substantially straight tear line for about 3 feet or more, or about 4 feet or more (including the entire length of the tubing, “L”). In some embodiments, the “peelability” or “tearability” can be achieved without any significant scoring or nicking. For example, in some embodiments, the score or nick across the cross-section of the tubing diameter has a length S that is less than about 1/10th the length L of the tubing to be peeled, less than about 1/25th the length L of the tubing to be peeled, less than about 1/50th the length L of the tubing to be peeled, or less than about 1/75th the length L of the tubing to be peeled. In some embodiments, such values can allow for complete peeling of the entire length of tubing and the peeled halves of tubing can be substantially equal in size (i.e., the tubing exhibits complete, straight, and/or even peeling along the entire length of the tubing.

The heat shrink tubes provided herein can be used in various ways. In some embodiments, they are advantageously used in catheter assembly processes, e.g., as a replacement for conventional (e.g., FEP) heat shrink tubes used as manufacturing aids to compress an underlying catheter pre-assembly during heating/reflow.

As such, the disclosed heat shrink (i.e., expanded) tube enables a catheter shaft to be manufactured by heating the heat shrink tube (sometimes referred to as a “manufacturing aid” or “fusing sleeve”) over top of an outer sheath tube (“jacket”) of a catheter shaft pre-assembly, inducing dimensional recovery of the heat shrink tube and allowing for reflow of the jacket material through a reinforcing component (where present) and bonding the jacket to the underlying liner. The selection of an appropriate heating temperature and time (also referred to herein as “heating profile” or “recovery profile”) for a particular heat shrink and outer jacket tube combination can vary substantially depending on the underlying catheter assembly components and the composition of the heat shrink tubing itself. In this way, expansion conditions, recovery ratio, and the recovery profile can be tailored to provide a heat shrink tube capable of reflowing an outer jacket for various types of catheter structures.

After production of the desired construct, the disclosed heat shrink tube is advantageously in some embodiments, able to be removed from the outer jacket without undue force, thus preventing damage to the underlying structure. In particular embodiments, the heat shrink tube is able to be peeled down its length (after a starting nick) without the need for skiving or cutting down its full length, further limiting the potential for damage to the outer jacket.

Further, in some embodiments, removal of the disclosed heat shrink tube allows it to release cleanly from underlying materials (e.g., an outer jacket). Releasing cleanly can refer to the lack of any visible residue on the underlying layer or on the heat shrink tubing. Releasing cleanly can alternatively (or additionally) refer to no visible deformation or damage of the underlying layer as a result of the removal process. In some embodiments, a heat shrink tubing can be considered to be cleanly released when, after removal, there is no visual detection of imperfections or material transfer e.g., on the outer surface of the jacket material.

Peelability and recovery of heat shrink tubes can also be characterized by thermo-mechanical testing with, for example, a TA instruments model Q800 Dynamic Mechanical Analyzer (DMA) equipped with film tension grips. For DMA peelability characterization, recovered tubes can be used. Specimens from the recovered tube can be sliced longitudinally along the centerline of the tube for a length of about 0.5 inches. Each half of the slit section can then be placed in the T-peel orientation in the DMA, with one section placed in the stationary grip and the other section placed in the movable grip. Specimens are tested at fixed amplitude using this method at 0.1 Hz, 1 Hz, and 10 Hz from −50° C. to 300° C. to determine tan 8 and storage modulus in this range. For DMA recovery characterization, a rectangular section can be cut radially from the expanded tube and placed in the tension grips. Individual test specimens are tested at fixed amplitude at a frequency of 1 Hz from −50° C. to 300° C. to determine tan 8 and storage modulus in this range.

With regard to the adhesion properties of the amorphous, non-fluorinated heat shrink materials, it may, in some embodiments, be important to evaluate adhesion between the heat shrink and underlying jacket substrate material to optimize lubricity and longitudinal peelability and, in some embodiments, ensure the ability to suitably remove the recovered heat shrink material. In certain embodiments, the het shrink tubings described herein comprise one or more additives to reduce adhesion and/or enhance peelability between the recovered heat shrink tubing material and the polymer substrate. In such embodiments, polymer resin(s) are blended prior to tube formation with one or more additives that reduce adhesion and enhance peelability between the heat shrink tubing material and the polymer substrate jacket material. Some such additives include, but are not limited to, ultrahigh molecular weight siloxane polymers, fumed silica materials, polyhedral oligomeric silsesquioxanes, liquid siloxanes, or any other siloxane materials can be blended, mixed, or incorporated into the heat shrink material prior to tube formation. The inclusion of one or more such additives may, in some embodiments, result in increased lubricity, a reduction of surface energy, and/or the prevention of adhesion or bonding to the underlying jacket material. Suitable methods for evaluating adhesion properties are generally known in the art.

### EXAMPLES

### Comparative Example 1

A commercially available FluoroPEELZ™ heat shrink tube was obtained (Zeus FluoroPEELZ™) and tested for dimensions, onset temperature, and the oven reflow test using the same procedures as Examples 1-8.

### Comparative Example 2

A commercially available poly(etherimide-siloxane) (PEI-Sil) copolymer resin (Sabic, SILTEM STM1600) was obtained in pellet form and dried in a membrane dryer at 220° F. until the resin moisture content was less than 0.02% by weight. The Vicat softening temperature was determined to be 167° C. The dried PEI-Sil resin was placed into a resin hopper dryer to prevent re-absorption of moisture before and during extrusion. The dried PEI-Sil pellets were melted and conveyed to an extrusion head by utilizing a single screw extruder having a barrel diameter of 1″, a screw rotation of 4.5-5.5 rpm, and barrel zone temperatures of 550-590° F. The extrusion head utilized a die temperature of 590-600° F., and an annular die set that provided a draw down ratio (DDR) of 27-28. After exiting the annular die set, the input tube passed through an enclosure to regulate cooling of the molten polymer and set the final tubular dimensions. An input tube having an average outer diameter of 0.091″ and an average total wall thickness of 0.013″ obtained using this process.

The prepared input tube was then expanded by pressurizing the inner diameter of the tube with compressed air as the input tube passed through a poly(tetrafluoroethylene) (PTFE) lined stainless steel hypodermic tube (also referred to herein as a “hypodermic tube” or “hypo tube” or just “hypo”) that resided within an infrared oven (also referred to herein as a “expansion oven” or just “oven”). The PTFE lined hypo tube serves as an expansion die and is used to restrict expansion of the input tube to a specified expanded diameter. The processing parameters of expansion air pressure applied to the ID of the input tubing, temperature of the pressurized expansion air applied to the ID of the input tubing, infrared oven temperature, and line speed were all adjusted to give a PEI-Sil heat shrink tube.

In particular, the heat shrink input tube of Comparative Example 2 was expanded using an oven temperature of 355-365° F., an expansion air pressure of 40 psi, and a line speed of 20 in/min.

A portion of the heat shrink tube produced in Comparative Example 2 was tested for Controlled Force Onset and the results have been summarized in Table 2 below.

### Example 1

The input tube prepared in Comparative Example 2 was utilized in Example 1. The prepared input tube was expanded using the same methods and conditions as Comparative Example 2, except that the expansion oven temperature was 295-305° F., and an expansion air pressure of about 136 psi was used.

A portion of the heat shrink tube produced in Example 1 was tested for Controlled Force Onset and the results have been summarized in Table 2 below.

### Example 2

The input tube prepared in Example 3 was utilized in Example 2. The prepared input tube was expanded using the same methods and conditions as Comparative Example 2, except that the expansion oven temperature was 255-265° F., and an expansion air pressure of about 240 psi was used.

A portion of the heat shrink tube produced in Example 2 was tested for Controlled Force Onset and the results have been summarized in Table 2 below.

### Example 3

The same commercially available PEI-Sil copolymer resin used in Comparative Example 2 was dried using the same method as Comparative Example 2 and was placed into a resin hopper dryer to prevent re-absorption of moisture before and during extrusion. The dried PEI-Sil pellets were melted and conveyed to an extrusion head by utilizing a single screw extruder having a barrel diameter of 1″, a screw rotation of 5.0-6.0 rpm, and barrel zone temperatures of 570-590° F. The extrusion head utilized a die temperature of 550-560° F., and an annular die set that provided a draw down ratio (DDR) of 22.5-23.5. After exiting the annular die set, the input tube passed through an enclosure to regulate cooling of the molten polymer and set the final tubular dimensions. An input tube having an average outer diameter of 0.107″ and an average total wall thickness of 0.021″ obtained using this process.

The prepared input tube was then expanded by pressurizing the inner diameter of the tube with compressed air as the input tube resided within a PTFE lined hypo and a forced air heating element (also referred to herein as a “thermal nozzle”) passed along the length of the hypodermic tube. The PTFE lined hypo tube serves as an expansion die and is used to restrict expansion of the input tube to a specified expanded diameter. The processing parameters of expansion air pressure applied to the ID of the input tubing, temperature of the pressurized expansion air applied to the ID of the input tubing, forced air heating element air temperature, forced air heating element air flowrate, forced air heating element traversal rate were all adjusted to give a PEI-Sil heat shrink tube.

In particular, the heat shrink input tube of Example 3 was expanded using a forced air heating element air temperature of 280-290° F., an expansion air pressure of 325 psi, forced air heating element air flowrate of 40 liters/minute, and a forced air heating element traversal rate of 0.5 mm/s.

A portion of the heat shrink tube produced in Example 6 was tested for Controlled Force Onset and the results have been summarized in Table 2 below.

### Example 4

The same commercially available PEI-Sil copolymer resin used in Comparative Example 2 was dried using the same method as Comparative Example 2 and was placed into a resin hopper dryer to prevent re-absorption of moisture before and during extrusion. The dried PEI-Sil pellets were melted and conveyed to an extrusion head by utilizing a single screw extruder having a barrel diameter of 1″, a screw rotation of 6.0-7.0 rpm, and barrel zone temperatures of 570-590° F. The extrusion head utilized a die temperature of 550-560° F., and an annular die set that provided a draw down ratio (DDR) of 10.5-11.5. After exiting the annular die set, the input tube passed through an enclosure to regulate cooling of the molten polymer and set the final tubular dimensions. An input tube having an average outer diameter of 0.081″ and an average total wall thickness of 0.008″ obtained using this process.

The prepared input tube was then expanded by pressurizing the inner diameter of the tube with compressed air as the input tube resided within a PTFE lined hypo and a forced air heating element (also referred to herein as a “thermal nozzle”) passed along the length of the hypodermic tube. The PTFE lined hypo tube serves as an expansion die and is used to restrict expansion of the input tube to a specified expanded diameter. The processing parameters of expansion air pressure applied to the ID of the input tubing, temperature of the pressurized expansion air applied to the ID of the input tubing, forced air heating element air temperature, forced air heating element air flowrate, forced air heating element traversal rate were all adjusted to give a PEI-Sil heat shrink tube.

In particular, the heat shrink input tube of Example 4 was expanded using a forced air heating element air temperature of 300-310° F., an expansion air pressure of 125 psi, forced air heating element air flowrate of 40 liters/minute, and a forced air heating element traversal rate of 0.5 mm/s.

A portion of the heat shrink tube produced in Example 4 was tested for Controlled Force Onset and the results have been summarized in Table 2 below.

### Example 5

The input tube prepared in Example 4 was utilized in Example 5. The prepared input tube was expanded using the same methods and conditions as Example 4, except that the forced air heating element air temperature of 280-290° F. and an expansion air pressure of 165 psi was used.

A portion of the heat shrink tube produced in Example 5 was tested for Controlled Force Onset and the results have been summarized in Table 2 below.

### Example 6

A commercially available poly(etherimide-siloxane) (PEI-Sil) copolymer resin (Sabic, SILTEM STM1700) was obtained in pellet form and dried in a membrane dryer at 220° F. until the resin moisture content was less than 0.02% by weight. The Vicat softening temperature was determined to be 180° C. The dried PEI-Sil resin was placed into a resin hopper dryer to prevent re-absorption of moisture before and during extrusion. The dried PEI-Sil pellets were melted and conveyed to an extrusion head by utilizing a single screw extruder having a barrel diameter of 1″, a screw rotation of 8.5-9.5 rpm, and barrel zone temperatures of 610-640° F. The extrusion head utilized a die temperature of 550-560° F., and an annular die set that provided a draw down ratio (DDR) of 8.5-9.5. After exiting the annular die set, the input tube passed through an enclosure to regulate cooling of the molten polymer and set the final tubular dimensions. An input tube having an average outer diameter of 0.076″ and an average total wall thickness of 0.013″ obtained using this process.

The prepared input tube was then expanded using the same method as Example 4 except that a larger expansion die was used. In particular, the heat shrink input tube of Example 6 was expanded using a forced air heating element air temperature of 375-385° F., an expansion air pressure of 250 psi, forced air heating element air flowrate of 40 liters/minute, and a forced air heating element traversal rate of 0.5 mm/s.

A portion of the heat shrink tube produced in Example 6 was tested for Controlled Force Onset and the results have been summarized in Table 2 below.

### Example 7

A commercially available poly(etherimide-siloxane) (PEI-Sil) copolymer resin (Sabic, SILTEM STM1500) was obtained in pellet form and dried in a membrane dryer at 220° F. until the resin moisture content was less than 0.02% by weight. The Vicat softening temperature was determined to be 78° C. The dried PEI-Sil resin was placed into a resin hopper dryer to prevent re-absorption of moisture before and during extrusion. The dried PEI-Sil pellets were melted and conveyed to an extrusion head by utilizing a single screw extruder having a barrel diameter of 1″, a screw rotation of 3.5-4.5 rpm, and barrel zone temperatures of 560-595° F. The extrusion head utilized a die temperature of 590-595° F., and an annular die set that provided a draw down ratio (DDR) of 10.0-11.0. After exiting the annular die set, the input tube passed through an enclosure to regulate cooling of the molten polymer and set the final tubular dimensions. An input tube having an average outer diameter of 0.076″ and an average total wall thickness of 0.013″ obtained using this process.

The prepared input tube was then expanded using the same method as Comparative Example 2 except that the expansion oven temperature was 215-225° F., and an expansion air pressure of about 150 psi and a line speed of 20 in/min was used.

A portion of the heat shrink tube produced in Example 7 was tested for Controlled Force Onset and the results have been summarized in Table 2 below.

### Example 8

A commercially available poly(etherimide-siloxane) (PEI-Sil) copolymer resin (Sabic, SILTEM STM1500) was obtained in pellet form and dried in a membrane dryer at 220° F. until the resin moisture content was less than 0.02% by weight. A commercially available poly(etherimide-siloxane) (PEI-Sil) copolymer resin (Sabic, SILTEM STM1600) was obtained in pellet form and dried in a membrane dryer at 220° F. until the resin moisture content was less than 0.02% by weight. The two PEI-Sil copolymers resins were pellet blended to a 25% Siltem STM1500 and 75% Siltem STM1600 weight ratio. The resulting PEI-Sil resin blend was placed into a resin hopper dryer to prevent re-absorption of moisture before and during extrusion. The dried PEI-Sil pellets were melted and conveyed to an extrusion head by utilizing a single screw extruder having a barrel diameter of 1″, a screw rotation of 6.0-9.0 rpm, and barrel zone temperatures of 570-590° F. The extrusion head utilized a die temperature of 550-560° F., and a monofilament die set that provided a draw down ratio (DDR) of 7.0-9.0. After exiting the monofilament die set, the solid strand passed through an enclosure to regulate cooling of the molten polymer and set the final monofilament dimensions. A strand pelletizer was then utilized to chop the monofilament strand into pellets. A PEI-Sil compound pellet having an average outer diameter of 0.110″ and an average length of 0.100″ was obtained using this process.

The Vicat softening temperature was obtained by calculating the arithmetic mean of the softening temperatures of the two resins and was determined to be 145° C.

The resulting PEI-Sil compound pellets were then dried in a membrane dryer at 220° F. until the resin moisture content was less than 0.02% by weight. The dried PEI-Sil compound was placed into a resin hopper dryer to prevent re-absorption of moisture before and during extrusion. The dried PEI-Sil compound pellets were melted and conveyed to an extrusion head by utilizing a single screw extruder having a barrel diameter of 1″, a screw rotation of 5.0-6.0 rpm, and barrel zone temperatures of 580-595° F. The extrusion head utilized a die temperature of 560-570° F., and an annular die set that provided a draw down ratio (DDR) of 11.0-12.0. After exiting the annular die set, the input tube passed through an enclosure to regulate cooling of the molten polymer and set the final tubular dimensions. An input tube having an average outer diameter of 0.091″ and an average total wall thickness of 0.013″ obtained using this process.

The prepared input tube was then expanded using the same method as Example 4. In particular, the heat shrink input tube of Example 9 was expanded using a forced air heating element air temperature of 270-280° F., an expansion air pressure of 253 psi, forced air heating element air flowrate of 40 liters/minute, and a forced air heating element traversal rate of 0.5 mm/s.

A portion of the heat shrink tube produced in Example 9 was tested for Controlled Force Onset and the results have been summarized in Table 2 below.

TABLE 1
Dimensional Attributes of the Heat Shrink Tubes of the Examples

| | | | | | After Heating | | | | | |
|---|---|---|---|---|---|---|---|---|---|---|
| | HS Recovery | Before Heating | | | (10 min) | | | Recovery Properties | | |
| | Onset | | Avg wt | | | Avg wt | | | | % Longitudinal |
| Example | Temperature (° C.) | ID (in) | (in) | Conc. | ID (in) | (in) | Conc. | RR | ΔID | Shrink [ΔL] |
| Comp. 1 | 81 | 0.1135 | 0.0068 | 88% | 0.0635 | 0.0108 | 96% | 1.79 | 44% | −1% |
| Comp. 2 | 180 | 0.1015 | 0.0085 | 89% | 0.0680 | 0.0125 | 92% | 1.49 | 33% | −20% |
| 1 | 127 | 0.1020 | 0.0087 | 92% | 0.0665 | 0.0119 | 92% | 1.53 | 35% | −13% |
| 2 | 98 | 0.0995 | 0.0101 | 84% | 0.0670 | 0.0136 | 95% | 1.49 | 33% | −15% |
| 3 | 108 | 0.1015 | 0.0159 | 91% | 0.0700 | 0.0210 | 91% | 1.45 | 31% | −15% |
| 4 | 134 | 0.1030 | 0.0058 | 76% | 0.0705 | 0.0084 | 91% | 1.46 | 32% | −14% |
| 5 | 122 | 0.1030 | 0.0056 | 70% | 0.0685 | 0.0083 | 90% | 1.50 | 33% | −12% |
| 6 | 187 | 0.1210 | 0.0046 | 78% | 0.0525 | 0.0126 | 94% | 2.30 | 57% | −36% |
| 7 | 89 | 0.1000 | 0.0073 | 77% | 0.0570 | 0.0136 | 90% | 1.75 | 43% | −27% |
| 8 | 111 | 0.1055 | 0.0090 | 90% | 0.0710 | 0.0140 | 94% | 1.49 | 33% | −14% |

TABLE 2
Controlled Force Testing Results

| | | | | HS Recovery | | | Average | | Reflowed | |
|---|---|---|---|---|---|---|---|---|---|---|
| | | | | Onset | | | Reflowed | Reflowed | Substrate | |
| | | | | Temperature | Polymer Substrate/ | | Substrate | Substrate | Defect | Contact |
| Example | RR | ΔL/ΔR | Δ_{VO} | (° C.) | Jacket Materials | Δ_{TO} | OD (in) | OD CoV | Rating | Clarity |
| Comp. | 1.79 | −0.02 | | 81 | A | 96.8 | 0.08376 | 0.000795 | 1 | PASS |
| 1 | | | | | B | 77.8 | 0.08349 | 0.003987 | 0 | PASS |
| | | | | | C | 53.8 | 0.08391 | 0.004871 | 0 | PASS |
| Comp. | 1.49 | −0.61 | −13 | 180 | A | −2.1 | 0.08535 | 0.004099 | 2 | PASS |
| 2 | | | | | B | −21.1 | 0.08438 | 0.001456 | 1 | PASS |
| | | | | | C | −45.1 | 0.08512 | 0.005949 | 2 | PASS |
| 1 | 1.53 | −0.38 | 40 | 127 | A | 50.8 | 0.08491 | 0.003121 | 2 | PASS |
| 2 | 1.49 | −0.46 | 69 | 98 | A | 79.9 | 0.08375 | 0.001129 | 1 | PASS |
| | | | | | B | 60.9 | 0.08377 | 0.001212 | 0 | PASS |
| | | | | | C | 36.9 | 0.08508 | 0.001357 | 1 | PASS |
| 3 | 1.45 | −0.48 | 59 | 108 | A | 69.8 | 0.08376 | 0.000904 | 1 | PASS |
| | | | | | B | 50.8 | 0.08409 | 0.000792 | 0 | PASS |
| 4 | 1.46 | −0.44 | 33 | 134 | A | 43.8 | 0.08356 | 0.000866 | 0 | PASS |
| 5 | 1.5 | −0.36 | 45 | 122 | A | 56.1 | 0.08323 | 0.002047 | 0 | PASS |
| | | | | | B | 37.1 | 0.08345 | 0.002422 | 0 | PASS |
| 6 | 2.3 | −0.64 | −7 | 187 | A | −8.7 | 0.08776 | 0.026549 | 3 | PASS |
| 7 | 1.75 | −0.63 | −11 | 89 | A | 89.5 | 0.08383 | 0.001330 | 1 | PASS |
| | | | | | B | 70.5 | 0.08421 | 0.000609 | 2 | PASS |
| | | | | | C | 46.5 | 0.08478 | 0.004237 | 2 | PASS |
| 8 | 1.49 | −0.43 | 34 | 111 | A | 66.8 | 0.08429 | 0.002302 | 1 | PASS |
| | | | | | B | 47.8 | 0.08459 | 0.003260 | 1 | PASS |
| | | | | | C | 23.8 | 0.08494 | 0.001544 | 2 | PASS |

The following polymer substrate/jacket materials were tested in Comparative Examples 1 and 2 as well as Examples 1-9 and noted in Table 3. Controlled Force Testing Results as (A) Nylon 12, 0.085″ ID, 0.005″ Wall-Dried, (B) Pebax 55D, 0.085″ ID, 0.005″ Wall-Dried, (C) Pebax 25D+ Lubricity Additive, 0.085″ ID, 0.005″ Wall-Dried. These polymer substrate jacket materials were selected to span soft to hard durometers and lower to higher melting temperature polymers conventionally used as jacket materials.

### Example 9

(A) 100% Siltem STM1500 Siltem; (B).98% Siltem STM 1500/2% Sinosil 1070; (C) 95% Siltem STM 1500/5% Sinosil 1070; (D) 98% Siltem STM 1500/2% liquid silozane. Sinosil 1070. For Example 9A-9D, heat shrink tube amorphous, non-fluorinated materials are prepared in a Brabender 3 piece Prep Mixer (Model 02-23-000.200) equipped with roller type blades. Siltem STM1500 is melt blended with Sinosil 1070, a 70/30 ratio high molecular weight siloxane polymer and fumed silica powder at loadings of 2 and 5% and form a plaque.

Alternatively, a 98% Siltem STM1500 and 2% liquid siloxane blended plaque is injected molded at 620° F. Various jacket materials are also pressed into plaques including Arkema PEBAX 2533, 3533, 5533, and 7233 as well as Avient Neusoft 842A at 400° F. Daikin Neoflon FEP NP-120 plaques are pressed at 650° F.

In some embodiments plaque dimensions are 3.5×3.5 inches and 0.04 inches thick. In some embodiments, the plaque dimensions are 3.5×2.5 inches and 0.12 inches thick. In some embodiments the liquid siloxane blend maintains the transparency of the Siltem STM1500 while mixing with other forms of siloxane additives and can result in opaque plaques.

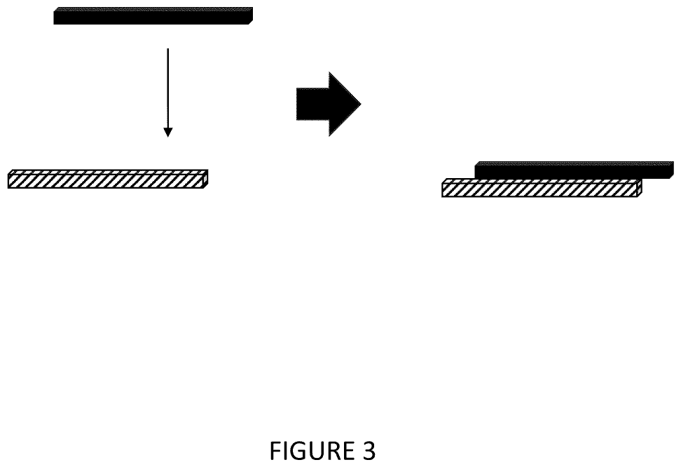

In some embodiments, samples for adhesion testing are prepared on a VWR Pro 10 x10 digital hot plate set at 220° C. Siltem strips are cut from the plaques and laid onto the hot plate followed by strips of a jacketing material as shown in FIG. 3. A 5 lb weight is placed on top of the samples and they are allowed to heat for 3 minutes then removed from the hot plate and allowed to cool to room temperature. A PTFE membrane sleeve is used to prevent sample adhesion to the hot plate and weight.

Adhesion between the heat shrink polymer materials 9A-D plaques and various polymer substrate jacket material plaques was evaluated qualitatively and quantitatively as described in Example 9. Adhesion results are shown in Table 3.

TABLE 3
Adhesion Results

| Example | FEP NP- | Pebax | Pebax | Pebax | Pebax | Neusoft |
|---|---|---|---|---|---|---|
| 9 | 120 | 72D | 55D | 35D | 25D | 842A |
| A | 0 | 0 | 0 | 1 | 2 | 2 |
| B | 0 | 0 | 0 | 0.5 | 1 | 1 |
| C | 0 | 0 | 0 | 0.5 | 1 | 1 |
| D | 0 | 0 | 0 | 0.25 | 0.5 | 0.5 |

Many modifications and other embodiments of the disclosure will come to mind to one skilled in the art to which this disclosure pertains having the benefit of the teachings presented in the foregoing description. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A heat shrink tubing for recovery over a polymer substrate, wherein the heat shrink tubing comprises at least one amorphous polymer resin,
wherein the heat shrink tubing exhibits a recovery temperature and a Vicat softening temperature and the polymer substrate exhibits a melting point such that:
a. a first delta (Δ_{TO}) between the melting point of the polymer substrate and an onset point of the recovery temperature of the heat shrink tubing is a positive value; and
b. a second delta (Δ_{VO}) between the Vicat softening temperature of the heat shrink tubing and the onset point of the recovery temperature of the heat shrink tubing is a positive value.

2. The heat shrink tubing of claim 1, wherein the at least one amorphous polymer resin is a non-fluorinated amorphous polymer.

3. The heat shrink tubing of claim 1, comprising no fluorinated polymer.

4. The heat shrink tubing of claim 1, that exhibits translucency or transparency.

5. The heat shrink tubing of claim 1, wherein the tubing comprises no more than one amorphous polymer resin.

6. The heat shrink tubing of claim 5, wherein the no more than one amorphous polymer is selected from the group consisting of poly(etherimide) (PEI), poly(imide) (PI), poly(carbonate) (PC), poly(siloxane), poly(sulfone) (PSU), poly(ethersulfone) (PESU), poly(phenylene sulfone) (PPSU), polyetherimide siloxane (PEI-Sil) copolymer, and copolymers and derivatives of any one or more thereof.

7. The heat shrink tubing of claim 1, wherein the tubing comprises two or more amorphous polymer resins.

8. The heat shrink tubing of claim 7, wherein the two or more amorphous polymer resins comprise:
a first amorphous polymer resin selected from the group consisting of poly(etherimide) (PEI), poly(imide) (PI), poly(carbonate) (PC), poly(siloxane), poly(sulfone) (PSU), poly(ethersulfone) (PESU), poly(phenylene sulfone) (PPSU), and copolymers, blends, and derivatives of any combination thereof; and
a second amorphous polymer resin selected from the group consisting of poly(etherimide) (PEI), poly(imide) (PI), poly(carbonate) (PC), poly(siloxane), poly(sulfone) (PSU), poly(ethersulfone) (PESU), poly(phenylene sulfone) (PPSU), polyetherimide siloxane (PEI-Sil) copolymer, and other copolymers, blends, and derivatives of any combination thereof.

9. The heat shrink tubing of claim 1, consisting essentially of the at least one amorphous polymer resin.

10. The heat shrink tubing of claim 1, further comprising one or more additives.

11. The heat shrink tubing of claim 1, wherein the tubing exhibits longitudinal peelability.

12. The heat shrink tubing of claim 11, wherein the longitudinal peelability does not require a physical score line or cut or embedded physical component to promote the longitudinal peelability or wherein the longitudinal peelability requires a physical score line, cut, or nick that is less than about 1/50th the length of the tubing.

13. The heat shrink tubing of claim 1, wherein the tubing has an average wall thickness of less than 0.02 inches.

14. The heat shrink tubing of claim 13, wherein the average wall thickness is 0.002 inches to 0.01 inches.

15. The heat shrink tubing of claim 13, wherein the average wall thickness is 0.004 inches to 0.0099 inches.

16. A catheter assembly comprising the heat shrink tubing of claim 1.

17. A construction, comprising a recovered heat shrink tubing comprising the heat shrink tubing of claim 1 recovered over the polymer substrate, wherein the polymer substrate is in reflowed form.

18. The construction of claim 17, wherein the polymer substrate exhibits a coefficient of variation (CoV) of its outer diameter (OD) of 0.005 or less.

19. The construction of claim 17, wherein the recovered heat shrink tubing exhibits a longitudinal to radial percent shrinkage heat shrink ratio of 0.5 or less with respect to the heat shrink tubing.

20. The construction of claim 17, wherein the recovered heat shrink tubing exhibits contact clarity through to the polymer substrate.

21. The construction of claim 17, wherein the recovered heat shrink tubing has an average wall thickness of 0.001 to 0.028 inches.

22. The construction of claim 21, wherein the average wall thickness is obtained when subjected to unrestricted recovery at 420° F. for five minutes.

23. A method of preparing a heat shrink tubing for recovery over a polymer substrate, comprising:
selecting a heat shrink tubing material comprising at least one amorphous polymer resin that exhibits a recovery temperature and a Vicat softening temperature; and
selecting a polymer substrate material that exhibits a melting point,
wherein the recovery temperature, Vicat softening temperature, and melting point are such that:
a. a first delta (Δ_{TO}) between the melting point of the polymer substrate and an onset point of the recovery temperature of the heat shrink tubing is a positive value; and
b. a second delta (Δ_{VO}) between the Vicat softening temperature of the heat shrink tubing and the onset point of the recovery temperature of the heat shrink tubing is a positive value.
